(19) United States
(12) Patent Application Publication
(10) Pub. No.: US 2018/0021286 A1
(43) Pub. Date: Jan. 25, 2018

(54) STABILIZED DIALKYL FUMARATE COMPOSITIONS

(71) Applicant: Enspire Group LLC, South Plainfield, NJ (US)

(72) Inventors: Abdul Rashid, Livingston, NJ (US); Zhang Julia Zhang, Scotch Plains, NJ (US); Minh Tran, Secaucus, NJ (US); Dahai Guo, Belle Mead, NJ (US); Yi Zeng, Hillsborough, NJ (US); Charchil Vejani, Maple Shade, NJ (US); Hengsheng Adam Feng, Cherry Hill, NJ (US)

(73) Assignee: Enspire Group LLC, South Plainfield, NJ (US)

(21) Appl. No.: 15/548,017

(22) PCT Filed: Jan. 29, 2016
(86) PCT No.: PCT/US16/15597

§ 371 Date: Aug. 1, 2017

Related U.S. Application Data
(60) Provisional application No. 62/110,852, filed on Feb. 2, 2015.

(51) Int. Cl. A61K 31/225; A61K 47/44; A61K 9/10; A61K 9/48; A61K 47/26; A61K 47/10; A61K 47/12

(52) U.S. Cl. CPC A61K 31/225; A61K 47/10; A61K 47/44; A61K 47/12; A61K 9/4808; A61K 9/4858; A61K 9/4866; A61K 9/4891; A61K 47/26; A61K 9/10

(57) ABSTRACT

Stabilized liquid, suspension or gel compositions containing a dialkyl fumarate and comprising a vehicle which serves as a protective base, a pharmaceutically acceptable surfactant, and a pharmaceutically acceptable acidulant, and methods of preparing such compositions are disclosed. Capsules and other dosage forms containing the stabilized liquid, suspension or gel compositions are also disclosed.

### CROSS-REFERENCE TO RELATED APPLICATIONS

This application claims the benefit of priority of U.S. Provisional Application No. 62/110,852, filed on Feb. 2, 2015, the entire disclosure of which is incorporated herein by reference.

### FIELD OF THE DISCLOSED SUBJECT MATTER

The presently disclosed subject matter relates to stabilized liquid, suspension or gel compositions containing a dialkyl fumarate, soft and hard shell capsules filled with the dialkyl fumarate compositions, other dosage forms containing such compositions, and methods of making same.

### BACKGROUND

Dialkyl fumarates (fumaric acid esters) have been recognized to provide effective oral therapy for psoriasis. Other diseases, including necrobiosis lipoidica, granuloma annulare, sarcoidosis, alopecia areata, cheilitis granulomatosa, recurrent oral aphthae, pityriasis rubra pilaris, annular elastolytic giant-cell granuloma, non-infectious chronic uveitis and multiple sclerosis (MS) have been found to respond to oral treatment with dialkyl fumarates, specifically dimethyl fumarate (DMF). DMF was shown to have a significant effect on relapse rate and time to progression of MS. DMF is believed to have immunomodulatory properties without significant immunosuppression. Long-term studies have established the safety of orally administered dialkyl fumarates without an increased risk of infections, malignancies, or significant long-term toxic effects.

Dialkyl fumarates are somewhat lipophilic (DMF has a Log P value of 0.74) and highly mobile in human tissue. As α,β-unsaturated electrophilic compounds, dialkyl fumarates are rapidly attacked by the nucleophilic detoxifying agent glutathione (GSH) in an in vivo Michael reaction. Reaction with other nucleophiles in a formulation of reactive excipients is also possible. Further, dialkyl fumarates are susceptible to ester hydrolysis, either in vivo or ex vivo. When administered orally, DMF does not survive long enough to be absorbed into blood without being attacked by GSH, although the monoester product of esterase-induced metabolism is more resistant to GSH.

In addition, dimethyl fumarate is a solid which sublimes under ambient conditions, providing physical challenges for formulating DMF.

Further, dimethyl fumarate has been found to be an allergic skin sensitizer at very low concentrations. Concentrations as low as 1 ppm can produce dermal allergic reactions.

This combination of physical properties, reactivity and sensitizing potential requires careful selection of pharmaceutical formulations when formulating dialkyl fumarates for oral administration. There continues to be a need for superior formulations which stabilize and isolate the dialkyl fumarates until delivered to the appropriate target within the human body.

### SUMMARY OF THE DISCLOSED SUBJECT MATTER

New formulations providing desirable protection and stabilization of the dialkyl fumarate active ingredient are provided herein.

In view of the above considerations, one approach to formulating dialkyl fumarates included isolating the active ingredient (AI) within a soft shell capsule (such as a softgel), a hard shell capsule, or other dosage form, and specifically stabilizing the AI in a suitable liquid, suspension or gel composition. Dimethyl fumarate (DMF) was used as a representative dialkyl fumarate.

A suitable vehicle which would serve as a protective base was sought for this composition in order to protect the dialkyl fumarate from the degradation reactions discussed above. It was discovered that edible natural waxes and oils, such as bees wax, vegetable oils, hydrogenated vegetable oils, diglycerides, triglycerides, and mixtures of two or more thereof provided a protective base for the composition. Alternatively a more hydrophilic vehicle/protective base was discovered to be poly(alkylene glycol)-based, with a mixture comprising poly(ethylene glycol)s being particularly useful.

Further, an optional solubilizing agent, also identified herein as a bioavailability enhancing agent, or generally as a pharmaceutically acceptable surfactant or emulsifier, was sought for the composition. Appropriate solubilizing agents were determined to be surfactants having a hydrophilic-lipophilic balance (HLB) of 4 or greater (e.g., 4 to 6; 6 to 8; 8 to 10; 10 to 12; 12 to 14; 14 to 16; 16 to 18; or 4 to 18).

Still further, a pH adjuster to maintain an acidic environment in the composition was sought. Without being bound by any particular theory, it is believed that an acidic environment favors stability of the dialkyl fumarate by preventing attack by any nucleophiles present in the composition. Suitable acids were found to be pharmaceutically acceptable organic acids, such as lactic acid, malic acid, citric acid, fumaric acid, ascorbic acid and tartaric acid.

The dialkyl fumarate dosage forms of the invention are distinguished over the prior art in at least the following ways:
(i) a liquid, suspension or gel formulation, including a high concentration of the active pharmaceutical ingredient (API); in combination with
(ii) delayed release, provided by a specific coating on the dosage form.

One aspect of the invention is directed to a liquid, suspension or gel composition comprising: a) 10-50% (e.g., 15 to 45%, 20 to 40%, 25 to 35%, 10%, 15%, 20%, 25%, 30%, 35%, 40%, 45%, or 50%) by weight of a dialkyl fumarate; bi) 30-75% (e.g., 40 to 70%, 50 to 65%, 30%, 40%, 45%, 50%, 55%, 60%, 65%, 70%, or 75%) by weight of a vehicle comprising one or more compounds selected from the group consisting of pharmaceutically acceptable waxes, solid aliphatic alcohols, vegetable oils, hydrogenated vegetable oils, and mixtures of two or more thereof; or bii) 30-85% (e.g., 35 to 80%, 40 to 75%, 45 to 70%, 50 to 65%, 30%, 30.2%, 35%, 40%, 45%, 50%, 55%, 60%, 65%, 70%, 75%, 80% or 85%) of a vehicle comprising one or more compounds selected from the group consisting of poly(ethylene glycols); c) 2-30% (e.g., 3-20%, 5 to 15%, 7 to 10%, 8-9%, 3%, 4%, 5%, 6%, 7%, 8%, 9%, 10%, 11%, 12%, 13%, 14%, 15%, up to 30%) by weight of at least one pharmaceutically acceptable surfactant; and d) 1-10% (e.g., 2 to 8%, 4 to 6%, 1%, 2%, 3%, 4%, 5%, 6%, 7%, 8%, 9%, or 10%) by weight of a pharmaceutically acceptable acidulant, based on the total weight of the composition. In some embodiments, the composition has one or more of the following features: (a) dialkyl fumarate and vehicle are present in a ratio of about 2:15 to about 10:9 (e.g., 1:7, 1:6, 1:5, 1:4, 1:3, 1:2, or 1:1) by weight; (b) dialkyl fumarate and surfactant are present in a ratio of about 2:3 to about 25:1 (e.g., 2:3, 1:1, 2;1, 3:1, 4:1, 5:1, 6:1, 10:1, 15:1, 20:1, or 25:1) by weight; and (c) dialkyl fumarate and an acidulant are present in a ratio of about 1:1 to about 50:1 (e.g., 1:1, 2;1, 3:1, 4:1, 5:1, 6:1, 10:1, 20;1, 30:1, 40:1, or 50:1) by weight.

In some embodiments of the composition the dialkyl fumarate is dimethyl fumarate. In some embodiments the dialkyl fumarate is the only active agent.

In some embodiments of the composition the vehicle comprises vegetable oils and waxes. The oils may comprise glycerides, including monoglycerides, diglycerides, and/or triglycerides. The vegetable oils can be selected from the group consisting of sunflower oil, soybean oil, safflower oil, canola oil, peanut oil, cottonseed oil, olive oil, sesame oil, corn oil and hydrogenated versions thereof. In one embodiment the vehicle consists essentially of 30-75% (e.g., 30%, 40%, 45%, 50%, 55%, 60%, 65%, 70% or 75%) by weight of sunflower oil, 0.5-5% (e.g., 0.5%, 1%, 2%, 3%, 4%, or 5%) by weight of a pharmaceutically acceptable wax, and 0.5-10% (e.g., 0.5%, 1%, 2%, 3%, 4%, 5%, 6%, 7%, 8%, 9%, or 10%) by weight of hydrogenated vegetable oil, based on the total weight of the composition. In other embodiments the vehicle is poly(alkylene glycol)-based, and can comprise 30-75% (e.g., 30%, 35%, 40%, 45%, 50%, 55%, 60%, 65%, 70% or 75%) by weight of a mixture of poly(ethylene glycol)s (PEGs). In one embodiment the mixture of PEGs comprises PEG 400 and PEG 1450.

In one embodiment of the composition the solubilizing or bioavailability enhancing agent is selected from the group consisting of pharmaceutically acceptable surfactants having an HLB of 4 or greater, and mixtures of two or more thereof. In one embodiment the surfactants are selected from the group consisting of fatty acid esters of polyoxylglycerides, fatty acid esters of polyoxylsorbitan, fatty acid esters of sorbitan, and mixtures of two or more thereof. In one embodiment the surfactants are selected from the group consisting of polyoxyl-35 hydrogenated castor oil, polyoxyl-40 hydrogenated castor oil (KOLLIPHOR® RH 40), polyoxyl-20 sorbitan monolaurate, polyoxyl-20 sorbitan monooleate, sorbitan esters (SPAN®), such as sorbitan monooleate (SPAN® 80), polyethoxylated sorbitan esters (TWEEN®), caprylocaproyl polyoxyl-8 glycerides, corn oil PEG-6 esters (LABRAFIL® M21125CS), lauroyl polyoxylglycerides (GELUCIRE® 44/14), and mixtures of two or more thereof. In one embodiment the surfactant consists essentially of 2-15% by weight of polyoxyl-40 hydrogenated castor oil based on the total weight of the composition. In one embodiment the surfactant comprises about 10% by weight of polyoxyl-40 hydrogenated castor oil and about 15% by weight of sorbitan monooleate based on the total weight of the composition.

In one embodiment of the composition the acidulant is selected from the group consisting of pharmaceutically acceptable organic acids and mixtures of two or more thereof. In one embodiment the acidulant is selected from the group consisting of lactic acid, malic acid, citric acid, fumaric acid, ascorbic acid, tartaric acid and mixtures of two or more thereof. In a preferred embodiment the acidulant is lactic acid. In one embodiment the acidulant consists essentially of 1-10% by weight of lactic acid based on the total weight of the composition.

One aspect of the invention is directed to a suspension formulation consisting essentially of: a) 10-50% by weight of dimethyl fumarate; b) a vehicle consisting essentially of: b1) 30-75% by weight of sunflower oil, b2) 0.5-5% by weight of bees wax, and b3) 0.5-10% by weight of hydrogenated vegetable oil; c) 2-15% by weight of polyoxyl-40 hydrogenated castor oil; and d) 1-10% by weight of lactic acid, based on the total weight of the composition.

As used herein, the transitional phrase “consisting essentially of” or “consists essentially of” limits the scope of a claim to the specified materials or steps and those that do not materially affect the basic and novel characteristics of the claimed invention. The term “consisting of” refers to compositions, methods, and respective components thereof as described herein, which are exclusive of any element not recited in that description of the embodiment.

For the purposes of the present application, “suspension formulation” or “suspension composition” indicates a composition which comprises a suspension of solid particles in a liquid vehicle, that is, a mixture in which active ingredient (AI) particles are dispersed throughout the vehicle. In order to form a suspension, the active ingredient must be insoluble or only partially soluble in the liquid vehicle at the concentration used in the formulation, and in the presence of the other formulation components.

Another aspect of the invention is directed to a suspension formulation consisting essentially of: a) 10-50% by weight of dimethyl fumarate; b) a vehicle consisting essentially of: b1) 30-75% by weight of PEG 400, and b2) 0.2-10% by weight PEG 1450; c) 1-15% by weight of polyoxyl-40 hydrogenated castor oil; and d) 1-10% by weight of lactic acid, based on the total weight of the composition.

One embodiment of the invention is directed to a suspension composition comprising a) about 10-50% by weight of dimethyl fumarate; b) a vehicle consisting essentially of about 30-75% by weight of a mixture of soybean oil, glyceryl distearate, and polyglyceryl-3 dioleate; ci) about 1-15% by weight of polyoxyl-40 hydrogenated castor oil; cii) about 1-20% by weight of sorbitan monooleate; and d) about 1-10% by weight of lactic acid, based on the total weight of the composition. In one embodiment the suspension composition comprises about 35% of DMF; about 4% of lactic acid; about 36% of a vehicle consisting essentially of soybean oil, glyceryl distearate, and polyglyceryl-3 dioleate; about 10% of polyoxyl-40 hydrogenated castor oil; and about 15% of sorbitan monooleate by weight. In a preferred embodiment the suspension composition comprises about 34.53% DMF; about 4.32% lactic acid; about 35.97% a vehicle consisting essentially of soybean oil, glyceryl distearate, and polyglyceryl-3 dioleate; about 10.07% polyoxyl-40 hydrogenated castor oil; and about 15.11% sorbitan monooleate by weight.

Another embodiment of the invention is directed to a suspension composition consisting essentially of a) about 10-50% by weight of dimethyl fumarate; b) a vehicle consisting essentially of about 30-75% by weight of a mixture of soybean oil, glyceryl distearate, and polyglyceryl-3 dioleate; ci) about 1-15% by weight of polyoxyl-40 hydrogenated castor oil; cii) about 1-20% by weight of sorbitan monooleate; and d) 1-10% by weight of lactic acid, based on the total weight of the composition. In one embodiment the suspension composition consists essentially of about 35% DMF; about 4% lactic acid; about 36% a vehicle consisting essentially of soybean oil, glyceryl distearate, and polyglyceryl-3 dioleate; about 10% polyoxyl-40 hydrogenated castor oil; and about 15% sorbitan monooleate by weight. In a preferred embodiment the suspension composition consists of 34.53% DMF; 4.32% lactic acid; 35.97% a vehicle consisting essentially of soybean oil, glyceryl distearate, and polyglyceryl-3 dioleate; 10.07% polyoxyl-40 hydrogenated castor oil; and 15.11% sorbitan monooleate by weight.

An additional embodiment of the invention is directed to a suspension composition comprising a) about 10-50% by weight of dimethyl fumarate; b) about 30-85% of a pharmaceutically acceptable vehicle; ci) about 1-15% by weight of polyoxyl-40 hydrogenated castor oil; cii) about 1-20% by weight of sorbitan monooleate; and d) about 1-10% by weight of lactic acid; based on the total weight of the composition. A further embodiment of the invention is directed to a suspension composition consisting essentially of a) about 10-50% by weight of dimethyl fumarate; b) about 30-85% of a pharmaceutically acceptable vehicle; ci) about 1-15% by weight of polyoxyl-40 hydrogenated castor oil; cii) about 1-20% by weight of sorbitan monooleate; and d) about 1-10% by weight of lactic acid; based on the total weight of the composition.

Yet another aspect of the invention is directed to a method of preparing a liquid, suspension or gel formulation of the invention, comprising the steps of:
(a) heating the vehicle to a temperature of 65±5° C. with stirring for a first period of time to obtain a first mixture;
(b) adding the surfactant into the first mixture with stirring at the same temperature to obtain a second mixture;
(c) cooling the second mixture to ambient temperature to obtained a cooled second mixture;
(d) adding the acidulant to the cooled second mixture with stirring at ambient temperature to obtain a third mixture;
(e) adding dialkyl fumarate to the third mixture with stirring at the ambient temperature to obtain a fourth mixture; and
(f) deaerating the fourth mixture, providing the liquid, suspension or gel formulation, the physical state of the formulation depending on the physical properties of the particular dialkyl fumarate.

Another aspect of the invention is directed to a softgel capsule comprising a soft gelatin or non-gelatin shell filled with any one of the above liquid, suspension or gel formulations. Another aspect of the invention is directed to a 2-piece hardshell capsule filled with any one of the above liquid, suspension or gel formulations. In one embodiment the gelatin of the soft gelatin capsule comprises bovine-, avian-, porcine-, marine- or vegetable-based gelatin, or a mixture of two or more thereof. In some embodiments the softgel capsule further comprises an enteric coating. In one embodiment enteric coating comprises a controlled release polymer. In one embodiment the controlled release polymer is an acid-resistant polymer.

### BRIEF DESCRIPTION OF THE DRAWINGS

FIG. 1 shows the dissolution profile of a softgel capsule of the invention (120 mg DMF) versus the Reference Listed Drug (RLD) TECFIDERA® capsule. Dissolution medium was pH 6.8 phosphate buffer.

FIG. 2 shows the dissolution profile of coated softgel capsules of the invention (240 mg DMF) in acid for 2 hours followed by pH 6.8 buffer.

FIG. 3 shows an in vitro/in vivo co-relationship (IVIVC) plot of Cₚ (ng/mL) versus time (min) for a DMF capsule of the invention.

### DETAILED DESCRIPTION OF PREFERRED EMBODIMENTS

Dialkyl fumarates have the structure of Formula (I):

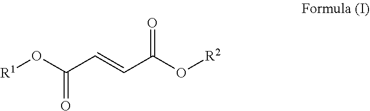

Dialkyl fumarates useful for the compositions of the present invention have R¹ and R² selected from the group consisting of C₁-C₁₈ alkyl. More particularly the C₃-C₁₈ alkyl groups can be linear, branched or cyclic. Preferably R¹ and R² are selected from C₁-C₄ alkyl, more preferably C₁-C₂ alkyl, and most preferably R¹ and R² are methyl. Although most commonly both alkyl groups are the same (that is, R¹═R²), R¹ and R² can be the same or different. In one embodiment, le and R² are independently selected from the group consisting of linear and branched C₁-C₁₈ alkyl. Optionally the alkyl groups can also contain 1-4 heteroatoms, such as oxygen, nitrogen or sulfur.

As noted above, a stabilized liquid, suspension or gel composition of the invention comprises a vehicle which serves as a protective base, optionally, at least one pharmaceutically acceptable surfactant, at least one pharmaceutically acceptable acidulant, and at least one dialkyl fumarate active ingredient. In some embodiments the stabilized liquid, suspension or gel composition consists essentially of a vehicle, at least one surfactant, at least one acidulant, and at least one dialkyl fumarate active ingredient. In other embodiments the stabilized liquid, suspension or gel composition consists of a vehicle, at least one surfactant, at least one acidulant, and at least one dialkyl fumarate active ingredient. The relative wt % ranges for these components, versus the total weight of the composition, has advantageously been found to be:

| Ingredient/Function | Example of ingredient | Range (wt %) |
|---|---|---|
| Vehicle/Protective Base | oil and/or wax | about 30-85 |
| Surfactant/Solubilizing | KOLLIPHOR ® RH 40 | about 0-30 |
| Agent | | |
| Acidulant | lactic acid | about 1-10 |
| Active Ingredient | dialkyl fumarate (e.g., | about 10-50 |
| | DMF) | |

For the purposes of the present invention, a “vehicle” which serves as a protective base for the disclosed compositions can comprise any pharmaceutically acceptable solvent or mixture of solvents which is compatible with the dialkyl fumarate active ingredient. With regard to DMF suspensions, for example, the vehicle can comprise any pharmaceutically acceptable solvent in which DMF is insoluble or partially soluble. Such a vehicle may comprise lipophilic solvents or excipients, such as medium chain triglycerides, long chain triglycerides, fatty acids, vegetable oils, or mixtures of two or more thereof. Lipophilic (hydrophobic) solvents and excipients can include pharmaceutically acceptable waxes and oils including bees wax, long-chain triglycerides, oleic acid, hydrogenated soybean oil, soy fatty acids, hydrogenated vegetable oil, d-α-tocopherol (vitamin e), corn oil, olive oil, corn oil mono-di-triglycerides, soybean oil, peanut oil, medium chain(c8/c10)mono- and di-glycerides, sesame oil, propylene glycol esters of fatty acids, medium-chain triglycerides, caprylic triglycerides derived from coconut oil or palm seed oil; fatty acids such as caprylic acid, capric acid lauric acid, myristic acid, palmitic acid, stearic acid, oleic acid, linoleic acid, γ-linoleic acid, ricinoleic acid, arachidic acid and behenic acid; peppermint oil, rapeseed oil, propylene glycol monolaurate, carnauba wax, paraffin wax, ethyl para-hydroxybenzoate, propylene glycol esters of fatty acids, lecithin, glyceryl monooleate, linoleoyl macrogolglycerides, cetyl alcohol, CRODACOL™ (a mixture of cetostearyl alcohol, stearyl alcohol and related compounds) and almond oil. The lipophilic excipient can also be any non-digestible pharmaceutical excipient.

Such a vehicle may also comprise hydrophilic solvents or excipients such as polyethylene glycols, polypropylene glycols, or mixtures thereof. Hydrophilic solvents and excipients can include polyethylene glycols (PEG 400, PEG 600, etc.), methoxy polyethylene glycol (MPEG 350, 550), diethyleneglycol monoethyl ether (TRANSCUTOL1®), tetrahydrofurfurylalcohol polyethylene glycol (GLYCOFUROL®), propylene carbonate, N-methyl-2-pyrrolidone (NMP), polyoxyethylene-polyoxypropylene copolymers (Poloxamers), propylene glycol, glycerin, and ethyl alcohol.

Alternatively such a vehicle may comprise a polymer, such as polyvinylpyrollidone, hydroxypropyl methyl cellulose (HPMC), hydroxypropyl cellulose (HPC); carrier molecules for the AI such as cyclodextrin; or surfactants/emulsifiers (for example oil-in-water emulsifiers) such as glyceryl distearate and polyglyceryl-3 dioleate.

It was discovered that edible natural waxes and oils, such as bees wax, vegetable oils, hydrogenated vegetable oils, and mixtures of two or more thereof provide an appropriate protective base vehicle for the composition. Alternatively a more hydrophilic protective base vehicle was discovered to be poly(alkylene glycol)-based, with a mixture of poly(ethylene glycol)s (PEGs) being particularly useful. Thus, the oil/wax vehicle can comprise non-polar and/or polar oils and/or waxes. Suitable polar oils include PEG oils, such as PEG 400, monoglycerides, and diglycerides such as glyceryl distearate. The mono- and di-glycerides can also be considered to be emulsifiers or surfactants. Suitable polar waxes include PEG waxes such as PEG 1450. Suitable non-polar oils include sunflower oil, soybean oil, hydrogenated vegetable oil and triglycerides. Suitable non-polar waxes include bees wax.

The optional solubilizing agent can include one or more of pharmaceutically acceptable emulsifying agents, pharmaceutically acceptable surfactants and related materials known in the pharmaceutical arts. In one embodiment the solubilizing agent is present in a concentration range of 0% to about 30% by weight, or alternatively 0% to about 15% by weight. Preferred solubilizing agents include polyoxyl-40 hydrogenated castor oil (KOLLIPHOR® RH 40), sorbitan monooleate (SPAN® 80) and polyglyceryl-3 dioleate (1,2,3-propanetriol, homopolymer, (9Z)-9-octadecenoate). In one preferred embodiment polyoxyl-40 hydrogenated castor oil is used as at least one of the solubilizing agents in a concentration range of about 1% to about 15% by weight. In another preferred embodiment polyoxyl-40 hydrogenated castor oil is used in a concentration range of about 1% to about 15% by weight and sorbitan monooleate is used in a concentration range of about 1% to about 20% by weight, based on the total weight of the composition. In one preferred embodiment the solubilizing agent comprises about 10% by weight of polyoxyl-40 hydrogenated castor oil and about 15% by weight of sorbitan monooleate based on the total weight of the composition.

The oil/wax and solubilizing agent can also be added as part of a commercial mixture, for example GELOIL® SC (available from Gattefossé SAS, Saint Priest Cédex, France), which is a nutraceutical ingredient consisting of an oily carrier mixture of refined GMO-free soybean oil (75 to <100%), the emulsifier glyceryl distearate (5 to 10%), and the emulsifier polyglyceryl-3 dioleate (1 to 5%).

As noted above, it is believed that a pH adjuster to maintain an acidic environment in the composition favors stability of the dialkyl fumarate by preventing attack by any nucleophiles which might be present in the final composition. Suitable acids were found to be pharmaceutically acceptable organic acids, such as lactic acid, malic acid, citric acid, fumaric acid, ascorbic acid and tartaric acid. In a preferred embodiment the acidulant comprises lactic acid. In another preferred embodiment the acidulant consists essentially of lactic acid. In yet another preferred embodiment the acidulant consists of lactic acid. The lactic acid can be L-lactic acid, D-lactic acid, racemic D/L-lactic acid, or any combinations thereof. Preferably, the acidulant is present in an amount that maintains the pH of the formulation in the range of 2-7.

In one embodiment the pH of the formulation is about 2 to about 3.5.

One aspect of the invention is directed to a liquid, suspension or gel composition comprising a dialkyl fumarate as the active ingredient, plus a combination of excipients selected to protect, stabilize and effectively deliver the active ingredient. The composition can be a solution or a suspension or a gel, depending on the physical properties of the active ingredient and excipients. One embodiment of the invention is directed to a liquid, suspension or gel formulation comprising: a) 10-50% by weight of a dialkyl fumarate; bi) 30-75% by weight of a vehicle comprising one or more compounds selected from the group consisting of bees wax, vegetable oils, hydrogenated vegetable oils, and mixtures of two or more thereof; or bii) 30-85% by weight of a vehicle comprising one or more compounds selected from the group consisting of poly(alkylene glycol)s; c) 1-30% by weight of at least one pharmaceutically acceptable surfactant; and d) 1-10% by weight of a pharmaceutically acceptable acidulant based on the total weight of the composition. In some embodiments, the composition has one or more of the following features: (a) dialkyl fumarate and vehicle are present in a weight ratio of about 2:15 to about 10:9; (b) dialkyl fumarate and surfactant are present in a weight ratio of about 2:3 to about 25:1; and (c) dialkyl fumarate and an acidulant are present in a weight ratio of about 1:1 to about 50:1. In one embodiment the composition has all of features (a), (b) and (c). In one embodiment the composition has features (a) and (b). In one embodiment the composition has features (a) and (c). In one embodiment the composition has features (b) and (c). In one embodiment the vehicle comprises bi). In another embodiment the vehicle comprises bii). In a preferred embodiment the vehicle bii) comprises poly(ethylene glycol)s; in a particularly preferred embodiment bii) comprises 30-75% by weight of PEG 400 and 0.2-10% by weight of PEG 1450. In another particularly preferred embodiment bii) consists essentially of 30-75% by weight of PEG 400 and 0.2-10% by weight of PEG 1450. In yet another particularly preferred embodiment bii) consists of 30-75% by weight of PEG 400 and 0.2-10% by weight of PEG 1450. In one embodiment bi) comprises 45-75% by weight of the composition; preferably 46-75%. Alternatively bi) can comprise 50-70%, 55-65% or about 60% of the composition by weight. In another embodiment bii) comprises 30-85% by weight of the composition, preferably 30.2-85%. Alternatively bii) can comprise 35-80%, 40-75%, 45-70%, 50-65% or about 55-60% of the composition by weight.

In some embodiments of the composition the dialkyl fumarate is dimethyl fumarate (Formula (I), R¹═R²═CH₃). In other embodiments the dialkyl fumarate can be monomethyl monoethyl fumarate (Formula (I), R¹═C₂H₅, R²═CH₃) or diethyl fumarate (Formula (I), R¹═R²═C₂H₅), or mixtures of various dialkyl fumarates. In some embodiments the dialkyl fumarate is the only active agent or ingredient.

In some embodiments of the composition the vegetable oils of the protective base vehicle are selected from the group consisting of sunflower oil, soybean oil, safflower oil, canola oil, peanut oil, cottonseed oil, olive oil, sesame oil, corn oil and hydrogenated versions thereof. In one preferred embodiment the vegetable oil is sunflower oil. In one embodiment the vegetable oil is hydrogenated vegetable oil, or a mixture of non-hydrogenated and hydrogenated vegetable oils and/or waxes. In one embodiment the vehicle consists essentially of 45-60% by weight of sunflower oil, 0.5-5% by weight of bees wax, and 0.5-10% by weight of hydrogenated vegetable oil, based on the total weight of the composition. Other acceptable vehicle components include fatty acid glycerides, including fatty acid triglycerides and/or fatty acid diglycerides, such as glyceryl distearate.

In some embodiments of the composition the poly(alkylene glycol)s are selected from the group consisting of poly(ethylene glycol)s, poly(propylene glycol)s and poly(butylene glycol)s. Preferably the poly(alkylene glycol)s are selected from the group consisting of poly(ethylene glycol)s, also known as PEGs, or poly(propylene glycol)s.

The solubilizing agent solubilizes the dialkyl fumarate active ingredient in the composition. It also serves to enhance the bioavailability of the active ingredient, and is generally a pharmaceutically acceptable surfactant or emulsifier. Therefore this component is referred to herein as a “solubilizing or bioavailability enhancing agent” or “solubilizing/bioavailability enhancing agent”, or simply as a “surfactant”. In one embodiment of the composition the solubilizing or bioavailability enhancing agent is selected from the group consisting of pharmaceutically acceptable surfactants and emulsifiers having an HLB of about 4 or greater, and mixtures of two or more thereof. Appropriate HLB values include, for example, about 4 to about 6; about 6 to about 8; about 8 to about 10; about 10 to about 12; about 12 to about 14; about 14 to about 16; about 16 to about 18; or about 4 to about 18. In one embodiment the surfactants are selected from the group consisting of fatty acid esters of polyoxylglycerides, fatty acid esters of polyoxylsorbitan, fatty acid esters of sorbitan, and mixtures of two or more thereof. In one embodiment the surfactants are selected from the group consisting of polyoxyl-35 hydrogenated castor oil, polyoxyl-40 hydrogenated castor oil, polyoxyl-20 sorbitan monolaurate, polyoxyl-20 sorbitan monooleate, caprylocaproyl polyoxyl-8 glycerides, LABRAFIL® M21125CS (HLB=9), GELUCIRE® 44/14 (HLB=11), sorbitan monooleate (SPAN® 80), and mixtures of two or more thereof. Polyoxyl-40 hydrogenated castor oil, also known as macrogol-glycerolhydroxystearate, is represented by the commercial product KOLLIPHOR® RH 40 (available from BASF). KOLLIPHOR® RH 40 is a nonionic solubilizer and emulsifying agent obtained by reacting one mole of hydrogenated castor oil with 40 moles of ethylene oxide. Polyoxyl-20 sorbitan monolaurate and polyoxyl-20 sorbitan monooleate, also known as polyoxyethylene (20) sorbitan monolaurate and polyoxyethylene (20) sorbitan monooleate, are commonly known as polysorbate 20 and polysorbate 80, respectively. Caprylocaproyl polyoxyl-8 glycerides, also known as caprylocaproyl macrogol-8 glycerides or PEG-8 caprylic-capric glycerides, is commercially available under the tradename LABRASOL® from Gattefosse. Corn oil PEG-6 esters (LABRAFIL® M21125CS) and lauroyl polyoxylglycerides (GELUCIRE® 44/14) are also available from Gattefosse. In some embodiments the surfactant consists essentially of 1-15% or 2-15% by weight of polyoxyl-40 hydrogenated castor oil based on the total weight of the composition. In other embodiments the surfactant is a combination of polyoxyl-40 hydrogenated castor oil and sorbitan monooleate (SPAN® 80) in a combined amount of about 1-30%, preferably about 1-25% by weight. In a preferred embodiment polyoxyl-40 hydrogenated castor oil is used in a concentration range of about 1% to about 15% by weight and sorbitan monooleate is used in a concentration range of about 1% to about 15% by weight, based on the total weight of the composition. In another preferred embodiment the surfactant comprises about 10% by weight of polyoxyl-40 hydrogenated castor oil and about 15% by weight of sorbitan monooleate based on the total weight of the composition.

In one embodiment of the composition the acidulant is selected from the group consisting of pharmaceutically acceptable organic acids and mixtures of two or more thereof. In one embodiment the acidulant is selected from the group consisting of lactic acid, malic acid, citric acid, fumaric acid, ascorbic acid, tartaric acid and mixtures of two or more thereof. In a preferred embodiment the acidulant is lactic acid. In one embodiment the acidulant consists essentially of 1-10% by weight of lactic acid based on the total weight of the composition.

One aspect of the invention is directed to a suspension formulation consisting essentially of a) 10-50% by weight of dimethyl fumarate; b) a vehicle consisting essentially of: b1) 45-60% by weight of sunflower oil; b2) 0.5-5% by weight of bees wax; and b3) 0.5-10% by weight of hydrogenated vegetable oil; c) 2-15% by weight of polyoxyl-40 hydrogenated castor oil; and d) 1-10% by weight of lactic acid, based on the total weight of the composition.

Another aspect of the invention is directed to a suspension formulation consisting essentially of a) 10-50% by weight of dimethyl fumarate; b) a vehicle consisting essentially of 30-75% by weight of a mixture of poly(alkylene glycol)s; c) 1-15% by weight of polyoxyl-40 hydrogenated castor oil; and d) 1-10% by weight of lactic acid, based on the total weight of the composition. In one embodiment the mixture of poly(alkylene glycol)s comprises poly(ethylene glycol)s (PEGs); preferably 30-75% by weight of PEG 400, and 0.2-10% by weight PEG 1450;

One embodiment of the invention is directed to a suspension composition comprising a) about 10-50% by weight of dimethyl fumarate; b) a vehicle consisting essentially of about 30-75% by weight of a mixture of soybean oil, glyceryl distearate, and polyglyceryl-3 dioleate; ci) about 1-15% by weight of polyoxyl-40 hydrogenated castor oil; cii) about 1-20% (or about 1-15%, or about 2-10%, or about 5-20%, or about 5-15%) by weight of sorbitan monooleate; and d) 1-10% by weight of lactic acid, based on the total weight of the composition. In one embodiment the suspension composition comprises about 35% DMF; about 4% lactic acid; about 36% vehicle consisting essentially of soybean oil, glyceryl distearate, and polyglyceryl-3 dioleate; about 10% polyoxyl-40 hydrogenated castor oil; and about 15% sorbitan monooleate by weight. In a preferred embodiment the suspension composition comprises about 34.53% DMF; about 4.32% lactic acid; about 35.97% vehicle consisting essentially of soybean oil, glyceryl distearate, and polyglyceryl-3 dioleate; about 10.07% polyoxyl-40 hydrogenated castor oil; and about 15.11% sorbitan monooleate by weight.

Another embodiment of the invention is directed to a suspension composition consisting essentially of a) about 10-50% by weight of dimethyl fumarate; b) a vehicle consisting essentially of about 30-75% by weight of a mixture of soybean oil, glyceryl distearate, and polyglyceryl-3 dioleate; ci) about 1-15% by weight of polyoxyl-40 hydrogenated castor oil; cii) about 1-20% by weight of sorbitan monooleate; and d) 1-10% by weight of lactic acid, based on the total weight of the composition. In one embodiment the suspension composition consists essentially of about 35% DMF; about 4% lactic acid; about 36% vehicle consisting essentially of soybean oil, glyceryl distearate, and polyglyceryl-3 dioleate; about 10% polyoxyl-40 hydrogenated castor oil; and about 15% sorbitan monooleate by weight. In a preferred embodiment the suspension composition consists essentially of about 34.53% DMF; about 4.32% lactic acid; about 35.97% vehicle consisting essentially of soybean oil, glyceryl distearate, and polyglyceryl-3 dioleate; about 10.07% polyoxyl-40 hydrogenated castor oil; and about 15.11% sorbitan monooleate by weight. In another preferred embodiment the suspension composition consists of 34.53% DMF; 4.32% lactic acid; 35.97% vehicle consisting essentially of soybean oil, glyceryl distearate, and polyglyceryl-3 dioleate; 10.07% polyoxyl-40 hydrogenated castor oil; and 15.11% sorbitan monooleate by weight.

An additional embodiment of the invention is directed to a suspension composition comprising a) about 10-50% by weight of dimethyl fumarate; b) about 30-85% of a pharmaceutically acceptable vehicle; ci) about 1-15% by weight of polyoxyl-40 hydrogenated castor oil; cii) about 1-20% by weight of sorbitan monooleate; and d) about 1-10% by weight of lactic acid; based on the total weight of the composition. A further embodiment of the invention is directed to a suspension composition consisting essentially of a) about 10-50% by weight of dimethyl fumarate; b) about 30-85% of a pharmaceutically acceptable vehicle; ci) about 1-15% by weight of polyoxyl-40 hydrogenated castor oil; cii) about 1-20% by weight of sorbitan monooleate; and d) about 1-10% by weight of lactic acid; based on the total weight of the composition.

Another aspect of the invention is directed to a method of preparing a liquid, suspension or gel formulation of the invention, comprising the steps of:
(a) heating the vehicle to a temperature of 65±5° C. with stirring for a first period of time to obtain a first mixture;
(b) adding the surfactant agent into the first mixture with stirring at the same temperature to obtain a second mixture;
(c) cooling the second mixture to ambient temperature to obtained a cooled second mixture;
(d) adding the acidulant to the cooled second mixture with stirring at ambient temperature to obtain a third mixture;
(e) adding dialkyl fumarate to the third mixture with stirring at the ambient temperature to obtain a fourth mixture; and
(f) deaerating the fourth mixture, providing the liquid, suspension or gel formulation, the physical state of the formulation depending on the physical properties of the dialkyl fumarate.
   One embodiment of the method consists essentially of steps (a) through (f). Another embodiment of the method consists of steps (a) through (f).

Another aspect of the invention is directed to a softgel capsule comprising a soft gelatin capsule filled with any one of the above liquid, suspension or gel formulations. Another aspect of the invention is directed to a 2-piece hardshell capsule filled with any one of the above liquid, suspension or gel formulations. In one embodiment the gelatin of the soft gelatin capsule comprises bovine-, avian-, porcine-, marine- or vegetable-based gelatin, or a mixture of two or more thereof. The gelatin formulation needs to be robust enough so that the capsules can withstand the next step of enteric coating. The gelatin formulation also needs to be compatible with active ingredient(s), excipients and enteric coating components. Thus, in some embodiments the softgel capsule further comprises an enteric coating. In one embodiment the enteric coating comprises a controlled release polymer. In one embodiment the controlled release polymer is an acid-resistant polymer. The enteric coating allows the capsule to pass through the stomach without releasing the active ingredient. Instead, the enteric coated softgels are designed to release their contents in the lower part of the duodenum or intestine, where the capsule wall is dissolved by the action of intestinal fluids and enzymes so that the active ingredient can pass into the blood vessels.

Thus, in one embodiment of the present invention, the softgel capsules are coated with a pharmaceutically acceptable pH-dependent entero-resistant polymer, allowing for a controlled release of the active pharmaceutical ingredient in the gastrointestinal tract. In particular, the release of the active substance in the stomach environment is minimized, whereby the majority of the amount of dialkyl fumarate is released in the intestines. The desired release rate in the intestines may be modulated by choosing the right combination of coating polymer(s), relative thickness of the coating layer surrounding the softgel and, optionally, by the inclusion of other excipients known to modify the release of the active substance. In one embodiment, the softgel is coated by at least one layer comprising a pharmaceutically acceptable pH-dependent entero-resistant polymer. A polymer is “pH-dependent entero-resistant” if the coating layer comprising it does not allow acidic gastric water to penetrate through but it allows the penetration of water to the dimethyl fumarate core (e.g., by dissolution, swelling, degradation etc.) at the essentially neutral pH of the intestines. In particular, a pH-dependent entero-resistant polymer suitable for purposes of the present invention is a polymer, which dissolves, swells or degrades at a pH of 4.5 or higher, preferably pH 5.0 or higher. In a typical embodiment, the polymer dissolves, swells or degrades at a pH in the range of from 4.5 to 7.0, preferably from 5.0 to 6.5. Non-limiting examples of suitable pH-dependent entero-resistant polymers useful as the coating material for purpose of the present invention include, alone or in combination, a polymethacrylate (for instance a copolymer of methacrylic acid and methyl methacrylate or a copolymer of methacrylic acid and ethyl aery late), hydroxypropyl methyl cellulose stearate, hydroxypropyl methyl cellulose acetate succinate (HPMC-AS), hydroxypropyl methyl cellulose phthalate (HPMC-P), polyvinyl acetate phthalate (PVAP), cellulose acetate phthalate (CAP) and shellac. Examples of suitable commercially available polymers of this kind are EUDRAGIT® L (such as EUDRAGIT® L30 D-55), EUDRAGIT® S and EUDRAGIT® FS and other brand-name equivalents thereof such as EASTACRYL® 30D and KOLLICOAT® 30. Suitable commercially available hydroxypropyl methyl cellulose phthalate polymers are hypromellose phthalate HP-55, hypromellose phthalate HP-555, and hypromellose phthalate HP-50. In one aspect, the temperature of coating (as measured on the product) does not exceed 40° C., preferably it does not exceed 30° C. More preferably, the coating temperature (as measured on the product) is between 20 and 25° C. The coating process advantageously is carried out by spraying the polymer dissolved or suspended in a coating liquid onto the surface of the particle(s). The coating liquid may be aqueous, alcoholic or a mixture thereof. Castor oil can be used as a plasticizer in the customary manner. Other conventional plasticizers, such as sorbitol can also be used.

The liquid, suspension or gel formulation can be encapsulated in soft gelatin shell or soft non-gelatin shell to form softgel capsules using a conventional rotary die process. Suitable soft gelatin shells may include (i) gelatin, 35-60% by weight; (ii) glycerin, 10-30% by weight; (iii) sorbitol, or anhydrized liquid sorbitol (such as ANIDRISORB®), 5-35% by weight; (iv) purified water, 20-50% by weight; and (v) artificial color, 0.0001-5% by weight.

The softgel capsules of the invention can also be prepared by other methods well known in the art. See e.g., P, K. Wilkinson et al., “Softgels: Manufacturing Considerations,” Drugs and the Pharmaceutical Sciences, 41 (Specialized Drug Delivery Systems); P, Tyle, Ed. (Marcel Dekker, Inc., New York, 1990) 409-449; F. S. Horn et at, “Capsules, Soft” Encyclopedia of Pharmaceutical Technology, vol. 2; J. Swarbrick and J. C. Boylan, eds. (Marcel Dekker, Inc., New York, 1990) pp. 269-284; M. S. Patel et al., “Advances in Softgel Formulation Technology,” Manufacturing Chemist, vol. 60, no. 7, pp. 26-28 (July 1989); M. S. Patel et al., “Softgel Technology,” Manufacturing Chemist, vol. 60, no. 8, pp. 47-49 (August 1989); R. F. Emerson, “Softgel (Soft Gelatin Capsule) Update,” Drug Development and Industrial Pharmacy (Interphex '86 Conference), vol. 12, no. 8 & 9, pp. 1133-1144 (1986); and W, R. Ebert, “Soft Elastic Gelatin Capsules: A Unique Dosage Form,” Pharmaceutical Technology, vol. 1, no. 5, pp. 44-50 (1977).

Without wishing to be bound by any particular theory, it is believed that the compositions, capsules and other dosage forms of the invention release their active ingredients (dialkyl fumarate, e.g., dimethyl fumarate) in vivo by an emulsification mechanism. The inventive liquid, suspension or gel formulations contain oil and surfactant, so that upon contact with body fluids the released formulations produce O/W (oil-in-water) emulsions in vivo. Emulsification removes lipophilic ingredients away from the active drug molecule and facilitates release of the drug in the hydrophilic environment found in vivo. Such emulsion formation may be an important factor for the release of the drug in such a way that it becomes bioavailable, as well as bio-equivalent to currently available solid dosage forms.

Thus, a liquid, suspension or gel composition, in one embodiment of the invention, releases dialkyl fumarate by a mechanism in which the formulation, upon interaction with an aqueous environment in vivo, forms an oil-in-water emulsion, thereby releasing the dialkyl fumarate and making it bioavailable. Liquid formulations have several advantages over solid formulations. Current marketed products which are mini tablets must be used within one month of opening the container, whereas softgel or hard-shell containing suspensions can be used longer after opening the container. This provides increased shelf life. Further a softgel is a tamperproof dosage form versus two-piece capsules.

Also, suspending the AI (which sublimes) into an anti-solvent (in which it does not dissolve, or only partially dissolves) significantly decreases exposure to outside environment, which in turn prevents sublimation or almost stops sublimation compared to solid tablets, powder, or other solid dosage forms. In one embodiment the final dosage form of the inventive liquid, suspension or gel formulation may be the liquid, suspension or the gel composition itself; that is, without further encapsulation.

Another aspect of the invention is directed to a unit dosage form suitable for oral administration to a patient; comprising any one of the above dialkyl fumarate compositions, where the unit dosage form is a capsule containing the composition and the capsule comprises a coating which does not release the composition until the pH of the surrounding, medium is 3.5 or higher. Preferably the dialkyl fumarate composition contains dimethyl fumarate (DMF). In one embodiment the capsule is a soft shell capsule. The soft shell capsule may be a gelatin or non-gelatin capsule. In another embodiment the capsule is a hard shell capsule. The hard shell capsule may be a gelatin or non-gelatin capsule. The non-gelatin hard shell capsule can comprise any edible plant-based gelatin-like material, for example, hydroxypropyl methyl cellulose (HPMC). In yet another embodiment the unit dosage form is the composition itself. In one embodiment the unit dosage form contains 120 or 240 mg of dimethyl fumarate. In another embodiment the unit dosage form contains 480 mg of dimethyl fumarate. In one embodiment dimethyl fumarate is the sole active ingredient.

Another aspect of the invention is directed to a method of treatment of multiple sclerosis, comprising administering to a patient in need thereof a treatment-effective dose of a dialkyl fumarate contained in any one of the above unit dosage forms. Preferably the dialkyl fumarate is dimethyl fumarate. In one embodiment the multiple sclerosis is a progressive form of multiple sclerosis.

Another aspect of the invention is directed to a method of treatment of patient having a condition characterized by at least one symptom chosen from neurodegeneration and neuroinflammation, comprising administering to a patient in need thereof a treatment-effective dose of a dialkyl fumarate contained in any one of the above unit dosage forms. Preferably the dialkyl fumarate is dimethyl fumarate.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

### EXAMPLES

### Example 1. Composition with Dimethyl Fumarate (DMF) as Active Ingredient

| Ingredient | Function | Range (%) |
|---|---|---|
| Sunflower Oil | Vehicle/ | 45-60 |
| Bees Wax | Protective Base | 0.5-5 |
| Hydrogenated Vegetable Oil | | 0.5-10 |
| KOLLIPHOR ® RH 40 | Surfactant | 2-15 |
| Lactic Acid | Acidulant | 1-10 |
| Dimethyl Fumarate | Active Ingredient | 10-50 |

A representative oil-based composition is provided below:

| | Lot# F00024P023 |
|---|---|
| Ingredient | (%) |
| Sunflower Oil | 52.40 |
| Bees Wax | 1.00 |
| Hydrogenated Vegetable Oil | 2.60 |
| KOLLIPHOR ® RH 40 | 8.00 |
| Lactic Acid | 4.00 |
| Dimethyl Fumarate | 32.00 |

### Stability

| | Test Lot#F00024P023 (Oil base) | | | | |
|---|---|---|---|---|---|
| | | 40° C./ | 40° C./ | 40° C./ | 40° C./ |
| | Initial | 75% RH | 75% RH | 75% RH | 75% RH |
| | Fill | 1 Week | 2 Weeks | 4 Weeks | 12 Weeks |
| Assay (%) | 94.7 | 95.2 | 92.4 | 98.4 | 96.2 |
| Total | <0.02 | <0.05 | <0.05 | <0.05 | <0.05 |
| Impurities (%) | | | | | |

All ingredients were mixed according to the procedure of Example 2.

### Example 2. Process for Preparation of Composition of Example 1

a) Weight all ingredients into a suitable Stainless Steel Container.
b) Add Sunflower Oil and Bees Wax into hydrogenated vegetable oil in the container. Heat the solution to 65° C.±5° C. Use Stainless Steel Propeller/High Shear Mixer to mix all ingredients for 40 minutes or until homogenized.
c) Add KOLLIPHOR® RH 40 into suspension (b) and continue to mix at 65° C.±5° C. for another 30 minutes or until homogenized.
d) Cool suspension from Step (c) to room temperature.
e) Add Lactic Acid into suspension of Step (d) and continue to mix for another 20 minutes or until homogenized
f) Add DMF powder into suspension of Step (e). Continue to mix for another 20 minutes.
g) Pass final suspension through milling machine with screen.
h) Deaerate the resulting suspension. Nitrogen blanket the final suspension container for storage.
   Encapsulation of the above suspension into a soft gelatin capsule (softgel) is accomplished by procedures known in the art. A nitrogen blanket is maintained during encapsulation. Optionally, the softgel capsules are then provided with an enteric coating consisting of hydroxypropyl methyl cellulose stearate with castor oil as plasticizer, in the customary manner,

### Example 3. Composition with Dimethyl Fumarate (DMF) as Active Ingredient, PEG-Based Formulation

| Ingredients | Function | Range (%) |
|---|---|---|
| PEG 400 | Vehicle/ | 30-75 |
| PEG 1450 Flake | Protective Base | 0.2-10 |
| KOLLIPHOR ® RH 40 | Surfactant | 1-15 |
| Lactic Acid | Acidulant | 1-10 |
| Dimethyl Fumarate | Active | 10-50 |

A representative PEG-based composition is provided below:

| | Lot# F00024P027 |
|---|---|
| Ingredients | (%) |
| PEG 400 | 57.00 |
| PEG 1450 Flake | 2.00 |
| KOLLIPHOR ® RH 40 | 5.00 |
| Lactic Acid | 4.00 |
| Dimethyl Fumarate | 32.00 |

### Stability

| Test | | | |
|---|---|---|---|
| Lot# F00024P027 | | Initial | 40° C./75% RH |
| (PEG base) | Initial Fill | Softgel Capsule | 1 month |
| Assay (%) | 99.8 | 97.6 | 98.3 |
| Total Impurities (%) | <0.05 | <0.05 | <0.05 |

All ingredients were mixed according to the procedure of Example 4.

### Example 4. Process for Preparation of Composition of Example 3

a) Weigh all ingredients into a suitable Stainless Steel Container.
b) Add PEG 400 and PEG 1450 into the container. Heat the solution to 65° C.±5° C. Use Stainless Steel Propeller/High Shear Mixer to mix all ingredients for 40 minutes or until homogenized.
c) Add KOLLIPHOR® RH 40 into suspension (b) and continue to mix at 65° C.±5° C. for another 30 minutes or until homogenized.
d) Cool suspension of Step (c) to room temperature.
e) Add Lactic Acid into suspension from Step (d) and continued to mix for another 20 minutes or until homogenized.
f) Add DMF into suspension of Step (e). Continue to mix for another 20 minutes.
g) Pass final suspension through milling machine with screen.
h) Deaerate the resulting suspension. Nitrogen blanket the final suspension container for storage.
   Encapsulation of the above suspension into a soft gelatin capsule (softgel) is accomplished by procedures known in the art. A nitrogen blanket is maintained during encapsulation. Optionally, the softgel capsules are then provided with an enteric coating consisting of hydroxypropyl methyl cellulose stearate with castor oil as plasticizer, in the customary manner.

### Example 5A. DMF 240 mg Suspension Composition

Dimethyl fumarate, 240 mg composition

| | | | % w/w |
|---|---|---|---|
| | Ingredients | Function | Range |
| 1 | Dimethyl fumarate (DMF) | Active | 10-50% |
| 2 | lactic acid | pH adjuster | 1-10% |
| 3 | GELOIL ® SC¹ | Combination | 30-85% |
| | | of vehicle/ | |
| | | emulsifiers | |
| 4 | Polyoxyl-40 hydrogenated castor oil | Surfactant | 1-15% |
| | (KOLLIPHOR ® RH 40) | | |
| 5 | sorbitan monooleate (SPAN ® 80) | Surfactant | 1-20% |

| | |
|---|---|
| Total | 100% |

¹GELOIL ® SC is a commercial mixture of refined soybean oil, glyceryl distearate and polyglyceryl-3 dioleate.

In one embodiment, the composition contains:

Dimethyl fumarate, 240 mg composition

| | Ingredients | % w/w | mg/cap |
|---|---|---|---|
| 1 | dimethyl fumarate (DMF) | 34.53 | 240 |
| 2 | lactic acid | 4.32 | 30 |
| 3 | GELOIL ® SC | 35.97 | 250 |
| 4 | Polyoxyl-40 hydrogenated castor oil | 10.07 | 70 |
| | (KOLLIPHOR ® RH 40) | | |
| 5 | sorbitan monooleate (SPAN ® 80) | 15.11 | 105 |

| | | |
|---|---|---|
| Fill Weight of capsule | 100.00 | 695 |

All ingredients were mixed according to the procedure of Example 6.

### Example 5B. DMF 120 mg Suspension Composition

Dimethyl fumarate, 120 mg composition

| | | | % w/w |
|---|---|---|---|
| | Ingredients | Function | Range |
| 1 | Dimethyl fumarate (DMF) | Active | 10-50% |
| 2 | lactic acid | pH adjuster | 1-10% |
| 3 | GELOIL ® SC¹ | Combination | 30-85% |
| | | of vehicle/ | |
| | | emulsifiers | |
| 4 | Polyoxyl-40 hydrogenated castor oil | Surfactant | 1-15% |
| | (KOLLIPHOR ® RH 40) | | |
| 5 | sorbitan monooleate (SPAN ® 80) | Surfactant | 1-20% |

| | |
|---|---|
| Total | 100% |

¹GELOIL ® SC is a commercial mixture of refined soybean oil, glyceryl distearate and polyglyceryl-3 dioleate.

In one embodiment, the composition contains:

Dimethyl fumarate, 120 mg composition

| | Ingredients | % w/w | mg/cap |
|---|---|---|---|
| 1 | dimethyl fumarate (DMF) | 34.53 | 120 |
| 2 | lactic acid | 4.32 | 15 |
| 3 | GELOIL ® SC | 35.97 | 125 |
| 4 | Polyoxyl-40 hydrogenated castor oil | 10.07 | 35 |
| | (KOLLIPHOR ® RH 40) | | |
| 5 | sorbitan monooleate (SPAN ® 80) | 15.11 | 52.5 |

| | | |
|---|---|---|
| Fill Weight of capsule | 100.00 | 347.5 |

All ingredients were mixed according to the procedure of Example 6.

### Example 6. Manufacturing Process for DMF Suspension Compositions

Note: The manufacturing process should be carried out in a closed environment and base temperature should be less than 30° C. before addition of the DMF since the AI sublimes. Deaerate the fill preparation tank at −48 to −70 cm Hg.
1. Melt polyoxyl-40 hydrogenated castor oil (KOLLIPHOR® RH 40) at 55° C. Weigh melted KOLLIPHOR® RH 40 accurately to suitable container equipped with mixer.
2. Turn off the heat.
3. Weigh accurately sorbitan monooleate (SPAN® 80) and add to the container containing KOLLIPHOR® RH 40. Mix at 600 rpm for 10-15 min.
4. Weigh accurately GELOIL® SC (mixture of refined soybean oil, glyceryl distearate and polyglyceryl-3 dioleate E475) and add to the container of step 3. Mix at 600 rpm for 10-15 min.
5. Weigh accurately lactic acid and add to the container of step 4. Mix at 600 rpm for 10-15 min.
6. Weigh dimethyl fumarate and transfer to the container of step 5. Transfer the container for homogenization. Homogenize the material at 3000 rpm using 80# mesh screen for 15 min. Scrape material from the wall of the container manually. Blanket the suspension with nitrogen.

### Example 7. Formulation Composition for Gelatin Mass

| | Ingredients | Th % |
|---|---|---|
| 1 | purified water USP | 32.0 |
| 2 | Sorbitol Sorbitan Solution NF | 24.8 |
| | (Sorbitol Special) | |
| 3 | Gelatin 180 Bloom Lime Bone NF | 43.2 |

All ingredients were mixed according to the procedure of Example 8.

### Example 8. Manufacturing Process for Gelatin Mass Preparation

1. Set up a gelatin preparation tank equipped with mixer and heat for a temperature gauge reading between 60° C. to 85° C.
2. Through the inlet valve of the gel preparation tank, add in the pre-weight quantity of Sorbitan Solution NF and purified water USP.
3. Start mixing and allow the liquid temperature to reach 60° C. to 85° C. 4. Once the temperature reaches 60° C. to 85° C. add through the opening Gelatin 180 Bloom NF to the gelatin preparation tank.
5. Apply vacuum (−37 to −62 cm Hg) until the foam rises to the maximum level in the gel preparation tank, then close the vacuum valve. Continue mixing while maintaining gelatin temperature at 70±5° C. for 20 minutes.
6. After 20 minutes mixing period, open vacuum valve all the way and control the rising foam level by throttling the vacuum valve open and close. Do not allow foam to enter the vacuum source. When the foam level is stabilized, continue deaeration with vacuum valve open for 20 minutes (vacuum of −37 to −62 cm Hg).
7. Check the gelatin mass for air bubble. If air bubble is still visible continue mixing for an additional 5 to 15 minutes until the gelatin mass is free of air bubble.

### Example 9A. Characterization of Composition from Development Batches to Evaluate Physical Stability; a) Content Uniformity

Content uniformity was assessed by taking samples of fill suspension from different locations in the vessel (top versus bottom) over the course of five days.

| T = 0 | | T = day 1 | | T = day 4 | | T = day 5 | |
|---|---|---|---|---|---|---|---|
| % Assay | % Assay | % Assay | % Assay | % Assay | % Assay | % Assay | % Assay |
| (Top) | (Bottom) | (Top) | (Bottom) | (Top) | (Bottom) | (Top) | (Bottom) |
| 102.2 | 101.1 | 101.6 | 103.1 | 95.1 | 102.7 | 98 | 97.31 |

Results and Discussion: % Assays of fill suspension at different sampling point were found to be uniform except for day 4. From the results, it can be concluded that sedimentation rate is very minimal and the suspension can be used with mixing prior to encapsulation.

### Example 9B. b) Freeze/Thaw Cycle

Freeze/thaw cycles were done by keeping fill suspension in a refrigerator and in a freezer for 48 hrs. After that it was removed and kept for an entire day at room temperature and the sedimentation ratio was measured. The same procedure was repeated for three consecutive cycles.

| | | Sedimentation | | | |
|---|---|---|---|---|---|
| Sedimentation Ratio | | Ratio | | Sedimentation Ratio | |
| (Cycle #1) | | (Cycle #2) | | (Cycle #3) | |
| Refrigerator | Freezer | Refrigerator | Freezer | Refrigerator | Freezer |
| None | None | 0.97 | 0.93 | 0.92 | 0.98 |

Results and Discussion: After freeze/thaw cycle, there was no significant change in the sedimentation which confirms the conclusion made from the data of content uniformity. The fill suspension can be used with mixing or agitation prior to encapsulation.

### Example 10A. Dissolution in pH 6.8 Phosphate Buffer of Hardshell Capsules Containing Fill Suspension Comprising Formulation F00024P054A1

% Release of dimethyl fumarate
(DMF) suspension

| | RLD¹ | |
|---|---|---|
| Time | TECFIDERA ® | |
| Point | 120 mg | PuraCap |
| (min) | Lot # P13007 | F00024P054A1 |
| 0 | 0.00 | 0.00 |
| 5 | 0.54 | 9.85 |
| 10 | 68.91 | 83.35 |
| 15 | 101.85 | 100.21 |
| 30 | 103.40 | 103.31 |
| 45 | 102.05 | 102.94 |
| 60 | 101.28 | 101.39 |
| 90 | 99.96 | 100.00 |

¹Reference Listed Drug

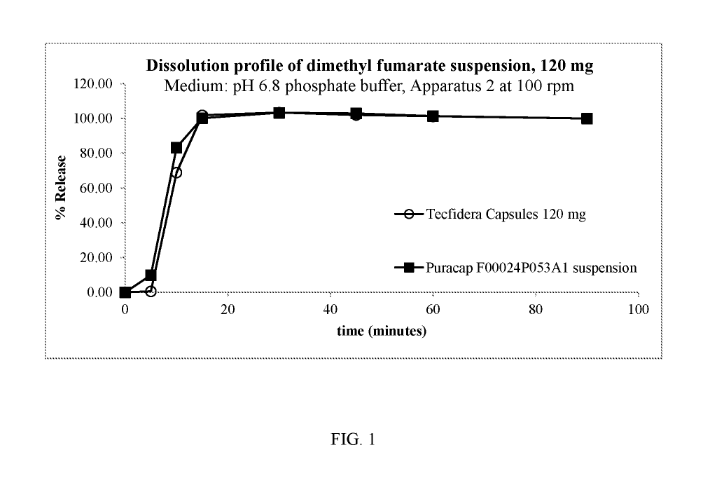

Dissolution profiles were created by plotting % release of active ingredient versus time. FIG. 1 shows the in vitro dissolution profile of PuraCap F00024P054A1-containing capsules plotted together with the Reference Listed Drug (RLD) TECFIDERA® capsules. The formulation is an oil-based suspension. The in vitro dissolution profile of PuraCap F00024P054A1-containing capsules was 100% in pH 6.8 buffer, which is similar to RLD, TECFIDERA®.

### Example 10B. Dissolution in Acid for 2 Hours Followed by pH 6.8 Phosphate Buffer of Coated Softgel Comprising Formulation F00024P054A1

| | Coating | | | | |
|---|---|---|---|---|---|
| Time (min) | wt: 5%¹ | 7.50%¹ | 10%¹ | 12.50¹% | RLD |
| 120 | 2.13 | 5.48 | 1.18 | 0.00 | 0.00 |
| (acid stage) | | | | | |
| 125 | 0.08 | 0.07 | 0.10 | 0.00 | 0.54 |
| 130 | 0.10 | 0.20 | 0.11 | 0.00 | 68.91 |
| 140 | 0.71 | 0.28 | 0.22 | 0.00 | 101.85 |
| 150 | 7.69 | 7.03 | 0.38 | 0.09 | 103.40 |
| 165 | 40.04 | 100.20 | 3.38 | 4.32 | 102.05 |
| 180 | 59.89 | 100.63 | 90.32 | 60.39 | 101.28 |

¹coating weight gains versus initial softgel weight

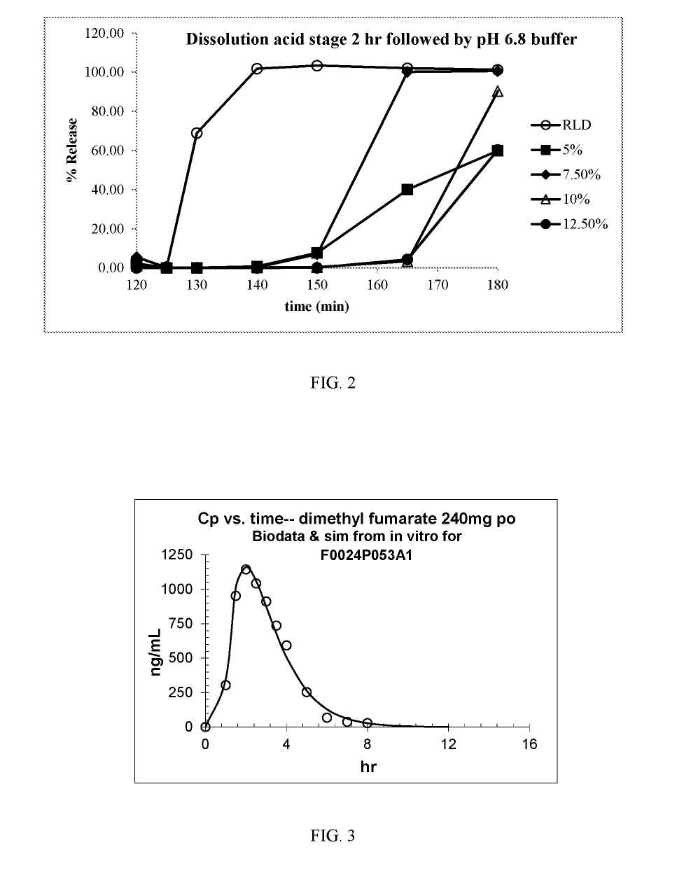

FIG. 2 shows the dissolution profiles.

### Example 11. In Vitro/In Vivo Co-Relationship (IVIVC)

| | RLD | | |
|---|---|---|---|
| | Clinical | RLD | F0024P054A1 |
| Time (hr) | Cₚ (ng/mL) | | |
| 0.0 | 0.0 | 0.0 | 0.0 |
| 1.0 | 304.3 | 276.3 | 349.7 |
| 1.5 | 952.4 | 938.1 | 1016.2 |
| 2.0 | 1144.0 | 1115.0 | 1162.3 |
| 2.5 | 1042.1 | 1051.6 | 1060.2 |
| 3.0 | 912.2 | 891.4 | 873.0 |
| 3.5 | 735.8 | 707.9 | 677.7 |
| 4.0 | 592.0 | 538.2 | 506.5 |
| 5.0 | 253.1 | 286.1 | 263.2 |
| 6.0 | 67.9 | 141.8 | 128.6 |
| 7.0 | 36.4 | 67.2 | 60.4 |
| 8.0 | 28.3 | 31.0 | 27.6 |
| 9.0 | | | 12.4 |
| 10.0 | | | 5.5 |
| 11.0 | | | 2.4 |
| 12.0 | | | 1.0 |

The IVIVC plot is shown as FIG. 3.

### Example 12A. Coating: Delayed-Release Coating Over Dimethyl Fumarate 120 Mg and 240 mg, Soft Gelatin Capsule

Materials, coating formula and function of the each ingredient:

| | | Coating |
|---|---|---|
| | Ingredients | solution |
| Ingredient | function | % w/w |
| EUDRAGIT ® | delayed-release | 52.235 |
| L30 D-55 | polymer | |
| dispersion (30% | | |
| solid) | | |
| PLASACRYL ® | anti-tacking | 15.686 |
| T20 (20% solid) | agent/plasticizer | |
| Triethyl Citrate | plasticizer | 0.785 |
| dyes | colorants | 0.158 |
| Purified Water | coating aid | 31.137 |
| | vehicle | |

| | |
|---|---|
| Total | 100.000 |

### Preparation of Coating Solution:

1. Weigh and transfer purified water into suitable container equipped with mixer.
2. Add triethyl citrate to container containing purified water and mix for 20 min at moderate speed.
3. Add dyes to step 2 and mix for 5-10 min at moderate speed.
4. Add PLASACRYL® T20 to step 3 and mix for 30 minutes at moderate speed.
5. Add EUDRAGIT® L30 D-55 dispersion to step 4 and mix for 1 hr at moderate speed.
   Pass resulting suspension through mesh #80 screen.

### Example 12B. Coating: Delayed-Release Coating Over Dimethyl Fumarate 120 Mg and 240 mg, Soft Gelatin Capsule

In one embodiment the dyes are (other ingredients same as Example 12A, above):

| | | Coating |
|---|---|---|
| | Ingredient | solution |
| Ingredients | function | % w/w |
| FD&C Blue #1 | colorant | 0.016 |
| Yellow #10 | colorant | 0.007 |
| White | colorant | 0.135 |
| dispersion | | |

### Preparation of Coating Solution:

1. Weigh and transfer purified water into suitable container equipped with mixer.
2. Add triethyl citrate to container containing purified water and mix it for 20 min at moderate speed.
3. Add Yellow #10, FD&C Blue #1 to step 2 and mix it for 5 min at moderate speed.
4. Add White dispersion to step 3 and mix it for 10 min at moderate speed.
5. Add PLASACRYL® T20 to step 4 and mix it for 30 minutes at moderate speed.
6. Add EUDRAGIT® L30 D-55 dispersion to step 5 and mix it for 1 hr at moderate speed.
7. Pass resulting suspension through mesh #80.

### Example 12C. Representative Coating Process

### Equipment for Coating Process:

Glatt Table top Lab scale coating pan, Model of GMPC-I Mini-Coater, fitted with 0.8 L perforated pan, having pan diameter of 7.5 inches and 11.0 inches depth. This equipment does not have the inbuilt facility to read the product temperature. The temperature is recorded from exhaust temperature or with infrared coating temperature gun. Drying process is accomplished through positive air pressure.

### Coating Gun:

Have the 1.0 mm bore diameter spray nozzle.
1. Pre-warm: Load the uncoated soft gelatin capsules and pre-warms the coating bed for 5 min or till the bed temperature is above 30° C. and record the pre-warming conditions and initial weight of capsules (50) in Coating parameters monitoring recording sheet.
2. Coating Suspension Spray: Once pre-warming is completed start spraying of Aqueous Delayed-Release coating suspension over the pre-wormed SG Capsules. Record Delayed-Release coating Spray processing conditions in Coating parameters Monitoring recording sheet. Intermittently take the sample and record the Ave Weight of coated capsules and record the observation of Visual Inspection and Coated Capsules weight gain. Adjust the conditions if required to prevent over wet of spray and record the same.
3. Spray the Delayed-Release coating suspension until targeted weight gain obtained. Stop the coating spraying of coating suspension once desired Delayed-Release coated polymer weight gain obtained. Reduce the pan speed and start the drying of Delayed-Release coated Capsules.
4. Testing: 10% delayed release weight gained coated Capsules tested for the Acid release by exposing coated capsules to 0.1N HCl media in dissolution vessel at 100 rpm speed using paddle for 2 hrs and followed by transferring the Acid exposed sample tested for pH 6.8 PBS Buffer, for drug release.

### Example 12D. Coating Process Conditions

Coating Process conditions from Glatt GMPC-1 mini cater

| | Spray Coating Parameter | Parameter value | |
|---|---|---|---|
| 1 | Inlet temperature ° C. | 45-55° | C. |
| 2 | Exhaust temperature ° C. | 30-40° | C. |
| 3 | Product temperature ° C. | Less than 38° | C. |
| 4 | Pattern air (bar) | 1.0 | |
| 5 | Atomizing air press (bar) | 1.5-2.0 | |
| 6 | Coating spray rate (g/min) | 1.0 to 2.0 | |
| 7 | Pan speed, RPM | 11-15 | |
| 8 | Pan load (g) | 300 | g |
| 9 | Pan volume (L) | 0.8 | L |
| 10 | Gun-to-bed distance | Approx 3-5 | inches |
| 11 | Drying product temperature after | 30-40° | C. |
| | coating (curing) ° C. | | |
| 12 | Drying time after coating (curing) | 1 | hr |

### Example 13. Stability at 40° C./75% RH

The composition F00024P054A1 was evaluated for stability at 40° C./75% RH.

| Test | Initial | 2 weeks | 4 weeks | 2 months | 3 months |
|---|---|---|---|---|---|
| Assay | 102.6 | 99.5 | 101.5 | 101.6 | 104.6 |
| Total related | <0.02% | 0.36 | 0.44 | 0.13 | 0.17 |
| compounds | | | | | |

The specific examples disclosed above are to be construed as merely illustrative, and not limitative of the remainder of the disclosure in any way whatsoever. Without further elaboration, it is believed that one skilled in the art can, based on the description herein, utilize the present invention to its fullest extent.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the present claims.

All publications cited herein are hereby incorporated by reference in their entirety.

What is claimed is:

1. A liquid, suspension or gel composition comprising:
a) about 10-50% by weight of a dialkyl fumarate;
bi) about 30-75% by weight of a vehicle comprising one or more compounds selected from the group consisting of pharmaceutically acceptable waxes, solid aliphatic alcohols, vegetable oils, hydrogenated vegetable oils, and mixtures of two or more thereof; or
bii) about 30-85% by weight of a vehicle comprising one or more compounds selected from the group consisting of of poly(ethylene glycols);
c) about 1-30% by weight of at least one pharmaceutically acceptable surfactant; and
d) about 1-10% by weight of a pharmaceutically acceptable acidulant;
based on the total weight of the composition.

2. The composition of claim 1 which has one or more of the following features:
(a) said dialkyl fumarate and said vehicle are present in a ratio of about 2:15 to about 10:9 by weight;
(b) said dialkyl fumarate and said surfactant are present in a ratio of about 2:3 to about 25:1 by weight; and
(c) said dialkyl fumarate and said an acidulant are present in a ratio of about 1:1 to about 50:1 by weight.

3-5. (canceled)

6. The composition of claim 1 where bii) comprises 30-75% by weight of PEG 400 and 0.2-10% by weight of PEG 1450.

7. (canceled)

8. The composition claim 1, wherein dialkyl fumarate is the only active ingredient, and said dialkyl fumarate is dimethyl fumarate.

9. (canceled)

10. The composition of claim 1, wherein said surfactant is selected from the group consisting of surfactants having an HLB of 4, or greater and mixtures of two or more thereof.

11-14. (canceled)

15. The composition of claim 1, wherein the vehicle consists essentially of 45-60% by weight of sunflower oil, 0.5-5% by weight of bees wax, and 0.5-10% by weight of hydrogenated vegetable oil, based on the total weight of the composition.

16. The composition of claim 1, wherein the surfactant consists essentially of 1-15% by weight of polyoxyl-40 hydrogenated castor oil based on the total weight of the composition.

17. The composition of claim 1, wherein the acidulant consists essentially of 1-10% by weight of lactic acid based on the total weight of the composition.

18. A method of preparing a liquid, suspension or gel composition of claim 1, comprising:
(a) heating the vehicle to a temperature of 65±5° C. with stirring for a first period of time to obtain a first mixture;
(b) adding the surfactant into the first mixture with stirring at the same temperature to obtain a second mixture;
(c) cooling the second mixture to ambient temperature to obtained a cooled second mixture;
(d) adding the acidulant to the cooled second mixture with stirring at ambient temperature to obtain a third mixture;
(e) adding dialkyl fumarate to the third mixture with stirring at the ambient temperature to obtain a fourth mixture; and
(f) deaerating the fourth mixture, providing the composition.

19. A softgel capsule comprising a soft gelatin or soft non-gelatin shell, or a 2-piece hardshell capsule filled with the liquid, suspension or gel composition of claim 1.

20-21. (canceled)

22. The capsule of claim 19, further comprising an enteric coating.

23. The capsule of claim 22, wherein said enteric coating comprises a controlled release polymer.

24-29. (canceled)

30. The suspension composition of claim 31, wherein dimethyl fumarate is present in about 34.53%, lactic acid is present in about 4.32%, the vehicle is present in about 35.97%, polyoxyl-40 hydrogenated castor oil is present in about 10.07%; and sorbitan monooleate is present in about 15.11% by weight.

31. A suspension composition comprising:
a) about 10-50% by weight of dimethyl fumarate;
b) about 30-85% of a pharmaceutically acceptable vehicle;
ci) about 1-15% by weight of polyoxyl-40 hydrogenated castor oil;
cii) about 1-20% by weight of sorbitan monooleate; and
d) about 1-10% by weight of lactic acid;
based on the total weight of the composition.

32. (canceled)

33. A softgel capsule comprising a soft gelatin or soft non-gelatin shell, or a 2-piece hardshell capsule filled with the suspension composition of claim 31.

34-35. (canceled)

36. The capsule of claim 33, further comprising an enteric coating.

37. The capsule of claim 36, wherein said enteric coating comprises a controlled release polymer.

38. (canceled)

39. A liquid, suspension or gel composition which releases dialkyl fumarate by a mechanism in which the formulation, upon interaction with an aqueous environment in vivo, forms an oil-in-water emulsion, releasing the dialkyl fumarate and making it bioavailable.

40. A unit dosage form suitable for oral administration to a patient, comprising the composition of claim 1, wherein said unit dosage form is a soft shell or hard shell capsule containing said composition and said capsule comprises a coating which does not release said composition until the pH of the surrounding medium is 3.5 or higher.

41-43. (canceled)

44. The unit dosage form of claim 40, containing 240 mg or 480 mg of dimethyl fumarate.

45. (canceled)

46. A method of treatment of multiple sclerosis, comprising administering to a patient in need thereof a treatment-effective dose of a dialkyl fumarate contained in the unit dosage form of claim 40.

47. (canceled)

48. A method of treatment of patient having a condition characterized by at least one symptom chosen from neurodegeneration and neuroinflammation, comprising administering to a patient in need thereof a treatment-effective dose of a dialkyl fumarate contained in the unit dosage form of claim 40.
